(19) United States
(12) Patent Application Publication
(10) Pub. No.: US 2006/0272078 A1
(43) Pub. Date: Dec. 7, 2006

(54) Apparatus and methodology to mitigate fogging on dual lens sports goggle

(76) Inventors: Riccardo Polinelli, Galliate Lombardo (IT); Dave Barton, Carlsbad, CA (US)

Correspondence Address: STETINA BRUNDA GARRED & BRUCKER, 75 ENTERPRISE, SUITE 250, ALISO VIEJO, CA, 92656, US

(21) Appl. No.: 11/376,890

(22) Filed: Mar. 16, 2006

Related U.S. Application Data
(63) Continuation of application No. 11/218,743, filed on Sep. 2, 2005.

(30) Foreign Application Priority Data
Oct. 29, 2004 (IT) MI2004A002082

(51) Int. Cl. A61F 9/02

(52) U.S. Cl. 2/436

(57) ABSTRACT

A dual pane lens assembly is provided which is adapted to be installed into a frame of a goggle. The dual pane lens assembly may include an outer lens, an inner lens positioned proximate the outer lens, and a gasket disposed between the outer and inner lens which adheres the inner lens to the outer lens, while further forming an air tight semi-annular space between the outer and inner lenses to mitigate against fogging. Dry dehumidified air may be disposed in the semi annular space to further mitigate against fogging. Also, the space between the lenses may be pressurized to a pressure between the atmospheric pressure of the goggle's expected use altitude and the atmospheric pressure of the goggle's assembly altitude such that distortion is minimized when the goggle is worn at the goggle's expected use altitude.

### CROSS-REFERENCE TO RELATED APPLICATIONS

This application is a continuation in part of U.S. application Ser. No. 11/218,743, filed on Sep. 2, 2005, which claims priority to pending Italian Patent Application No. M12004A002082, filed on Oct. 29, 2004, the disclosures of which are expressly incorporated herein by reference.

### STATEMENT RE: FEDERALLY SPONSORED RESEARCH/DEVELOPMENT

Not Applicable

### BACKGROUND

1. Field of the Invention

The present invention relates to sports goggles. In particular, the present invention relates to sports goggles which utilize dual pane lens assemblies for inhibiting the formation of fog on the lenses of the goggles.

2. Background of the Invention

Goggles have long been worn as eye protection in various sports, such as snow skiing, snowboarding, snowmobiling, motorcycling, auto racing, off-roading, ATV's, ski diving, mountain biking or other types of physical activities where wind and/or environmental debris, such as snow, mud, dirt, etc., may be projected toward the participant's eyes. As a means to protect the eyes, while also providing clear vision and a large field of view, the typical goggle includes a frame which positions a large lens in front of the eyes. The lens is positioned about an inch or so in front of the eyes, therefore, creating a protected open space around the eyes of the wearer. The frame of the goggle assembly is also maintained around the eye region of the wearer by a headband.

One problem which has yet to be solved with respect to sports goggles is the effective elimination of the formation of fog on the inside lens surface of the goggles. The fogging of goggles is typically caused by (1) moisture within the protected open space and (2) a difference in temperature within the protected open space and the outside environment. The source for the difference in temperature and moisture is typically generated from the wearer of the goggle. That is to say, the wearer is typically participating in an activity which is physically exhausting. As the wearer's heartbeat increases, the body heats up and begins to perspire, even in the vicinity of the eyes. Moisture is introduced within the protected open space and a temperature differential forms between the outside environment and the protected open space. The temperature differential condenses the moisture introduced into the protected open space onto the inside surface of the lens resulting in fog.

Various attempts have been made in an effort to inhibit the formation of fog inside goggles. For example, there are a variety of features which may be utilized or incorporated into a goggle to help prevent the formation of fog. Following is a brief discussion on the features which are now typically used in various combinations to reduce the formation of fog in sport goggles.

One of the most common features for inhibiting fog is the inclusion of vent paths in the lens. The vent paths are disposed proximately below an upper edge of the outer lens wherein one vent path is disposed above a left eye region of the lens and another vent path is disposed above a right eye region of the lens and wherein the vent paths are adapted to allow air to circulate into and out of the goggles.

Other attempts to inhibit the formation of fog inside the goggles have included coatings or “anti-fog” treatments applied to the lens surfaces. The treatments are designed to inhibit fog formation. Once again, although such an approach has been effective to a limited degree, it still has not been a complete solution to the fogging problem.

Another attempt to solve the fogging dilemma has included the use of dual paned lens assemblies. Dual paned lens assemblies utilize two lenses, instead of one. The concept of dual lenses is similar to that of a dual pane window. The space between the two lenses maintains a buffered temperature which helps to mitigate against the formation of fog on the inside lens surface of the inner lens. To further prevent fogging on the lens surfaces of the two lenses facing each other (i.e., outer surface of inner lens and inner surface of outer lens), the space between the two lenses is typically sealed off from the environment with a foam gasket. Unfortunately, as the goggle is repeatedly transported between sea level and snowboarding altitudes, the pressure within the space between the two lenses repeatedly fluctuates stressing the foam gasket and ultimately causing a leak such that the space between the two lenses is not completely sealed off from the environment. Moisture is introduced via the leak into the space between the two lenses causing fog on an inner surface of the outer lens and an outer surface of the inner lens.

Also, while the space between the two lenses is completely sealed off from the environment, a pressure differential exist between the space between the two lenses and the atmospheric pressure at snowboarding altitudes which cause distortion of objects viewed through the goggle thereby impairing the wearer's visibility through the lenses. To mitigate against distortion, air is permitted to enter and escape the space between the two lenses to equalize the pressure of the space between the two lenses and the ambient pressure as the goggle is transported between sea level and snow levels. Unfortunately, when air is introduced into the space between the two lenses, moisture is also introduced into the space between the two lenses which promotes fogging on the outer surface of the inner lens and the inner surface of the outer lens.

Prior art goggles have attempted to resolve the distortion problem but have been unsuccessful.

Accordingly, there is a need in the art for an improved dual lens goggle.

### BRIEF SUMMARY

A goggle is disclosed herein which addresses the problems identified above as well as problems identified below and known in the art.

According to a first embodiment of the goggle, a dual pane lens assembly is provided which is adapted to be installed into a frame. The dual pane lens assembly may include an outer lens; an inner lens positioned proximate the outer lens; and a gasket (e.g., polyurethane-based glue, etc.) disposed between the outer and inner lenses and about peripheries thereof. The gasket may form an air tight seal between the inner and outer lenses. Also, the gasket may adhere the inner lens to the outer lens, while further forming a semi-annular space between the outer and inner lenses.

According to an aspect of the goggle, the semi-annular space may be pressurized such that the pressure within the semi-annular space is about the atmospheric pressure of the goggle's expected use altitude when assembled such that transporting the goggle to the goggle's expected use altitude equalizes the pressure within the semi-annular space to about the atmospheric pressure of the goggle's expected use altitude to reduce or eliminate distortion due to pressure differences in the semi-annular space and the atmospheric pressure.

The pressure within the semi-annular space may be pressurized to three different levels when assembled. First, the semi-annular space may be pressurized to a pressure greater than the atmospheric pressure of the goggle's expected use altitude but less than the atmospheric pressure of the goggle's assembly altitude. Second, the semi-annular space may be pressurized to a pressure equal to about the atmospheric pressure of the goggle's expected use altitude. Third, the semi-annular space may be pressurized to a pressure less than the atmospheric pressure of the goggle's expected use altitude.

According to another aspect of the goggle, vents may be included in the frame for inhibiting fog. The frame vents allow the hotter air trapped inside the goggle to escape or migrate out of the protected open space such that the air within the protected open space becomes more equalized or normalized to the outside environment.

Furthermore, in another aspect of the goggle, a plurality of recessed portions formed in the upper edge of the outer lens and a recessed portion formed in a nose piece area of the outer lens are adapted to be received by the goggle frame. Also, in another aspect of the goggle, the outer lens is optically corrected and fabricated from polycarbonate material, a technology marketed as Accurate Radius Cuvature, or ARC®. Moreover, in another aspect of the goggle, at least one of the outer and inner lenses is coated with anti-fog coating.

Other exemplary embodiments and advantages of the goggle may be ascertained by reviewing the present disclosure and the accompanying drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front perspective view of an exemplary sports goggle which is adapted to accept a dual lens assembly;

FIG. 2 is a side perspective of the goggle of FIG. 1;

FIG. 3 is a front view perspective of the dual lens assembly adapted to be installed into the goggle of FIGS. 1 and 2;

FIG. 4 is a rear perspective view of the dual lens assembly of FIG. 3;

FIG. 5 is an upper rear perspective view of the dual lens assembly with a gasket interposed between inner and outer lenses of the dual lens assembly, the inner and outer lenses define a semi-annular space and a needle is inserted into the gasket;

FIG. 6 is an exploded perspective view of the dual lens assembly, the dual lens assembly having a gasket disposed between the inner and outer lenses; and

FIG. 7 is a chart which shows the targeted pressurization point relative to altitude.

### DETAILED DESCRIPTION

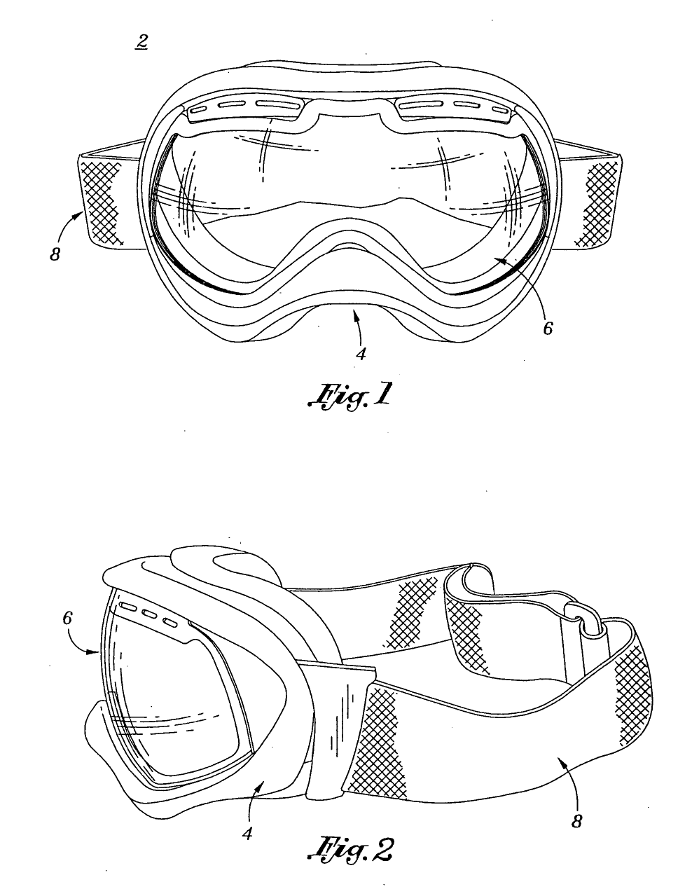

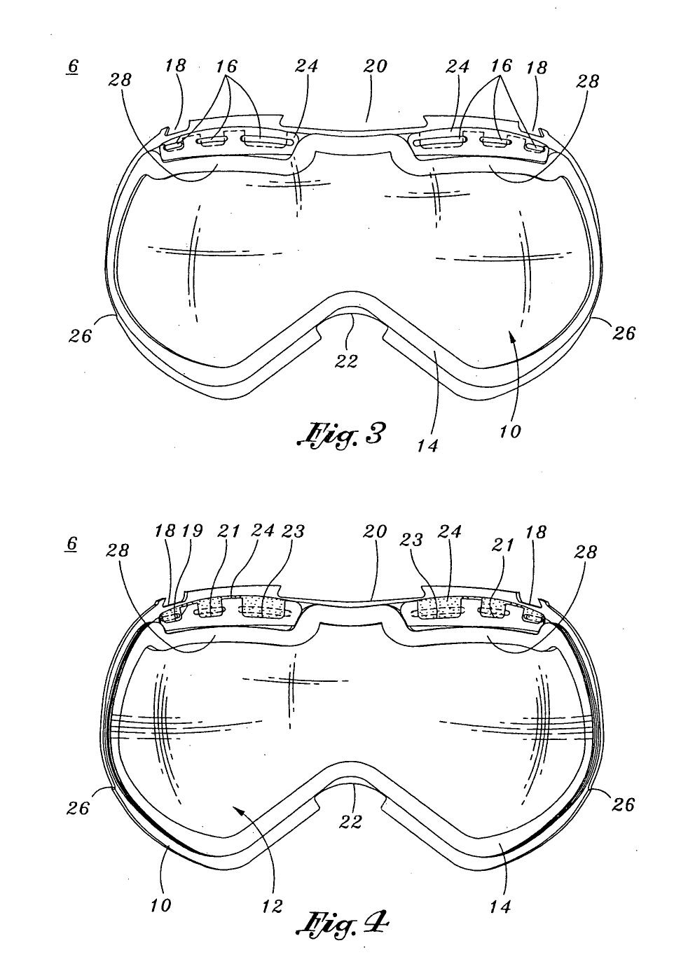

Referring now to the drawings which are for the purpose of illustration and not limiting this disclosure, FIG. 1 is a front perspective of an exemplary sports goggle 2 which is adapted to accept a dual lens (or dual pane) assembly 6. FIG. 2 is a side perspective view of the goggle of FIG. 1. The goggle assembly 2 may include a frame assembly 4 adapted to accept the dual lens assembly 6. Also, a headband 8 is typically attached to both sides of the frame assembly 4 for securing the goggle 2 to the wearer's head.

The frame assembly 4 may incorporate various features to mitigate against fogging, provide comfort to the user, etc. For example, the frame assembly 4 may utilize The Scoop® Frame Venting System, which helps to force air to circulate behind the lens assembly 6 to mitigate against fogging as disclosed in U.S. Pat. Nos. 5,601,668; 5,801,805; 5,898,468; and 6,050,684, the disclosures of which are expressly incorporated herein by reference.

The frame assembly 4 may be made from a variety of materials and processes known in the art. For instance, the frame assembly 4 may be made from thermoplastic, thermoset, polymers, composites and/or metal.

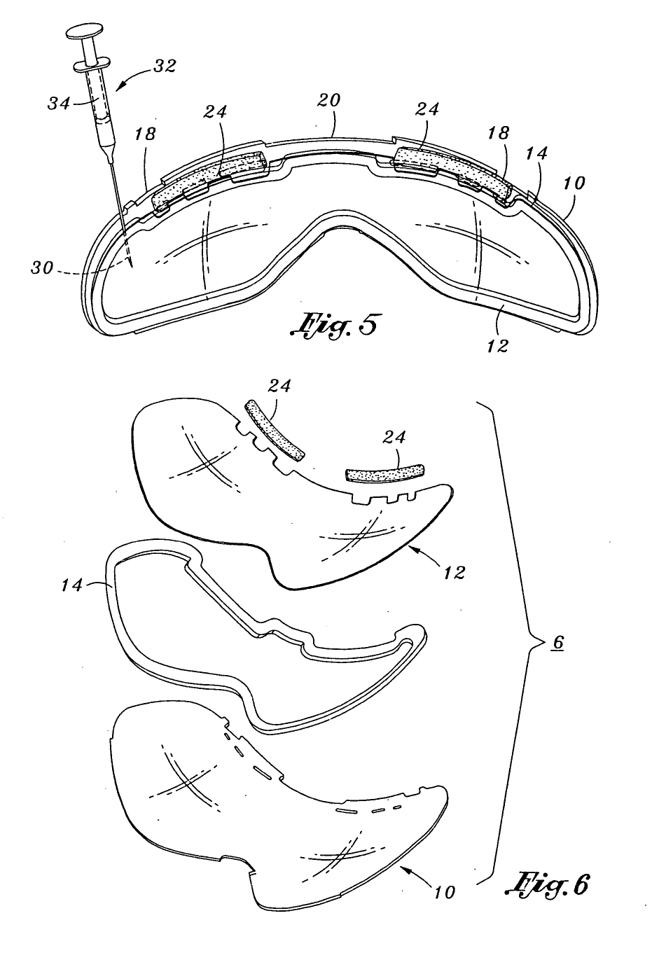

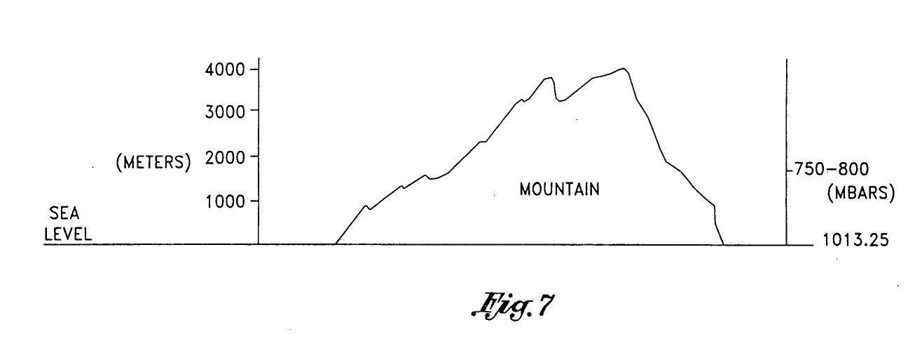

FIGS. 3 through 6 show a variety of views and perspectives of the dual lens assembly 6 adapted to be installed into the goggle 2 shown in FIGS. 1 and 2. In particular, FIG. 3 is a front view perspective of a dual lens assembly 6; FIG. 4 is a rear perspective view of the dual lens assembly 6; FIG. 5 is an upper rear perspective view of the dual lens assembly 6; and FIG. 6 is an exploded perspective view of the dual lens assembly 6.

The following paragraphs will now describe the parts, materials and structural details of which the exemplary dual lens assembly 6 is composed thereof. The dual lens assembly 6 utilizes a pair of lenses 10, 12 in a dual pane orientation (see FIG. 6). In particular, the dual lens assembly 6 utilizes an outer lens 10 and an inner lens 12. The lenses 10, 12 may be formed to have spherical shapes, cylindrical shapes, toroidal shapes or any other form or shape known to be used in lens design. Preferably, the shape of the outer lens 10 is formed to match the shape of inner lens 12, thereby, forming a semi-annular space between both lenses 10, 12.

The lenses 10, 12 may be fabricated from polycarbonate material, propionate cellulose or cellulose acetate. Preferably, the outer lens 10 is fabricated from polycarbonate material, and the inner lens 12 is fabricated from polycarbonate material, propionate cellulose, PC or cellulose acetate. Further, each lens 10, 12 may be designed to become thinner as it moves away from the optical center to mitigate against distortion. For example, the lenses 10, 12 may be lenses sold under the trademark ARC (Accurate Radius Curvature).

The lenses 10, 12 may also preferably be treated with various coatings, such as anti-scratch hardcoats, to make the lenses 10, 12 scratch resistance. Furthermore, the lenses 10, 12 may also be preferably treated with anti-fog coating, to help inhibit the formation of fog on the lenses 10, 12. Also, the lenses 10, 12 may be ARC® Polarized Lenses which are designed to diffuse blinding glare that bounces off of flat surfaces such as water and pavement. ARC® Polarized Lenses accomplish this by utilizing an Advanced Polarization Filter which is bonded between two layers of ARC® polycarbonate.

Moreover, the lenses 10, 12 may be tinted. For example, the lens tint options may include, but is not limited to the following: grey which is good for general-purpose lenses which offer true color perception; bronze which sharpens contrast and increases depth perception; clear which is used for low light conditions; green/grey which is considered a good general-purpose lens; high-intensity yellow which brightens low level light conditions; orange which sharpens contrast and which is used in flat light conditions; bronze with gold which is the same tint as bronze and which also includes a gold mirror coating; grey with silver which is the same as grey and which also includes a silver mirror coating; bronze with silver which is the same as bronze and which also includes a silver mirror coating; and grey polarized or bronze polarized which diffuses blinding glare that bounces off of flat surfaces such as water and pavement.

As shown in FIG. 3, the periphery of the outer lens 10 includes a plurality of recesses 18, 20 and 22 adapted to be received by the frame assembly 4. In particular, a pair of first recessed portions 18 are disposed in the left and right upper edge regions of the lens 10 near the temple regions. A second larger recessed portion 20 is further disposed in the center of the upper edge region of the lens 10. Also, in the nose region of the lens 10 a third recessed portion 22 is formed. Additionally, a pair of notches 26 is formed in the lower temple area of the outer lens 10. Furthermore, the outer lens 10 includes a plurality of longitudinal ports 16 which are disposed through the outer lens 10. One series of the ports 16 are positioned just below the upper edge of the outer lens 10 above the left eye region, while another series of ports 16 are positioned just below the upper region of the outer lens 10 above the right eye region. The longitudinal ports 16 are provided as a circulation means allowing air into the protected open space defined by the space between the eyes of the wearer and the inner lens 12.

As shown in FIG. 4, the perimeter shape of the inner lens 12 conforms to the perimeter shape of the outer lens 10, except, the perimeter shape of the inner lens 12 is slightly smaller (about ⅛ to about 3/16 inch) than the perimeter shape of the outer lens 10. The inner lens 12 may include a plurality of recessed portions 19, 21 and 23 which work with the longitudinal ports 16 of the outer lens 10 to provide vents/inlets which act as circulation means allowing air into the protected open space. The plurality of recesses 19, 21 and 23 are positioned directly behind the series of ports 16 of the outer lens 10. One series of the recessed portions 19, 21 and 23 is positioned just below the upper edge of the inner lens 12 above the left eye region, while another series of recessed portions 19, 21 and 23 is positioned just below the upper region of the outer lens 10 above the right eye region. More particularly, a pair of fourth recessed portions 19 are disposed in the left and right upper edge regions of the lens 12 near the temple regions. A second slightly larger fifth recessed portion 21 is further disposed inboard towards the center of the upper edge region of the lens 12. Also, a sixth recessed portion 23 is further disposed inboard of the fifth recessed portions towards the center of the upper edge region of the lens 12. The function of the plurality of recesses 19, 21 and 23 will be discussed in greater detail later in the specification.

To adhere both lenses 10, 12 together while also forming a seal between the outer and inner lenses 10, 12, a gasket 14 (see FIGS. 5 and 6) may be disposed between the lenses 10, 12. The gasket 14 may be a polyurethane based glue or any material for forming an airtight permanent seal. Although the goggle is discussed in relation to polyurethane based glues, it is also contemplated that other types of gasket materials may be used such as silicone based glue or rubber, a polyurethane-based rubber, or the like. The polyurethane based glue may be provided in a pasty form or a liquefied form. The polyurethane based glue may be applied to the peripheries of the lenses 10, 12. Once the polyurethane based glue has hardened, then a hermetic seal is formed between the lenses 10, 12. The hardened polyurethane based glue may have the characteristic of being self-sealing. For example, if the hardened polyurethane based glue is perforated by means of a needle or point having a small diameter, the hardened polyurethane based glue has the ability to close up again and restore the hermetic seal. Solvents and reticulating agents may be added to the polyurethane based glue.

The aforementioned polyurethane based glue has been selected at least for the reason that they exhibit an extremely low air and moisture permeability which forms an air and moisture tight seal between the lenses 10, 12. Also, as the goggle is repeatedly transported from sea level to snow levels, the polyurethane based glue does not degrade like the prior art foam gasket. The semi annular space continues to be completely sealed off from the environment. The air tight seal is maintained between the lenses 10, 12.

Preferably, the two lenses 10, 12 are machine-joined with the gasket 14 to assure production consistency. Also, preferably, the polyurethane based glue is machine applied to the lenses 10, 12 to form a machine applied “drool” type gasket 14.

To join the two lenses 10, 14 with a gasket 14, an activating treatment is carried out on the surface of the outer lens 10 intended to make contact with the polyurethane based glue. If the inner lens 12 is fabricated from polycarbonate, the activating treatment is also carried out on the surface of the inner lens 12 intended to make contact with the polyurethane based glue. The activation treatment modifies the molecular links and facilitates adhesion between the polyurethane based glue and the lenses 10, 12 to form the gasket 14.

As shown in FIGS. 4 and 6, the gasket 14 may be disposed proximate the outer peripheral edges of both lenses 10, 12. In particular, as shown in FIG. 4, the gasket 14 is disposed proximate the peripheral edge of the inner lens 12. More particularly, the gasket 14 may be disposed about 1/16 inch to about 3/16 inch away from the peripheral edge of the outer lens 10. When the outer lens 10 is sandwiched over the gasket 14 with the inner lens 12, the gasket 14 forms a seal between both lenses 10, 12. Preferably, the distance between the two lenses 10, 12 is uniform across the entire lens. For example, the inner lens may be about 3 mm gapped away from the outer lens.

Also, as is shown in FIG. 3, the gasket 14 may have a pair of offset and lowered regions 28 which are positioned lower than the recessed portions 19, 21 and 23 of the inner lens 12. The offset and lowered regions 28 also are positioned lower than the longitudinal ports 16. As a result of the configuration of the seal/gasket 14, a vent/circulation path is establish from the longitudinal ports 16 disposed on the outer lens 10 through the recessed portions 19, 21 and 23 of the inner lens 12 into the protected open space between the wearer's face and the goggle lens assembly 6. Additionally, as is shown in FIGS. 3 through 6, a foam insert 24 may be installed into the area which defines the vent/circulation path for preventing excessive moisture from entering the vent/circulation path.

Another aspect of the goggle 2 is that dry dehumidified air may be disposed within the semi-annular space. A method of disposing dry dehumidified air within the semi-annular space is to evacuate the air from the semi-annular space and introduce dry dehumidified air into the semi-annular space. For example, as shown in FIG. 5, a thin needle 30 of a syringe 32 may be pierced through the gasket 14 and into the semi-annular space. A plunger 34 (shown in dashed lines) of the syringe 32 may be retracted to evacuate the air from the semi-annular space. At this point, the semi-annular space may have a vacuum therewithin. The needle 30 may be removed from the gasket 14. The hole through the gasket 14 formed by the needle 30 may self seal such that environmental air is not introduced back into the semi-annular space. A needle 30 of a syringe 32 having a reservoir filled with dry dehumidified air may then be pierced into the gasket 14 at the same or different location. The dry dehumidified air within the reservoir may be introduced into the semi-annular space by pushing the plunger 34 forward. Minimal or no condensation will occur on the lens surfaces facing each other despite a temperature difference between the outside temperature and the temperature within the semi-annular space because only a negligible amount of moisture remains within the semi-annular space.

Another aspect of the goggle 2 is that a pressure within the semi-annular space may be set during assembly to be greater than the atmospheric pressure of the altitude of the goggle's expected use but less than the atmospheric pressure of the goggle's assembly altitude. For example, if the goggle 2 is expected to be used at an altitude between about twenty four hundred meters to about thirty three hundred meters (e.g., Mammoth Mountain skiing elevations), then the pressure within the semi-annular space may be set at assembly to an atmospheric pressure of an altitude of about two thousand (2000) meters. When the goggle is transported to or worn at the goggle's expected use altitude, the pressure within the semi-annular space approaches the atmospheric pressure of the goggle's expected use altitude to minimize a pressure difference between the pressure within the semi-annular space and the atmospheric pressure for reducing or eliminating distortion of objects viewed through the goggle.

The examples provided herein are for the purpose of illustration and not limitation. Although the examples above and below set the pressure within the semi annular space greater than the atmospheric pressure of the goggle's expected use altitude but less than the atmospheric pressure of the goggle's assembly altitude, it is also contemplated that the pressure within the semi-annular space may be set at assembly equal to about the atmospheric pressure of the goggle's expected use altitude (e.g., about twenty four hundred meters to about thirty three hundred meters). Alternatively, it is contemplated that the pressure within the semi-annular space may be set at assembly less than the atmospheric pressure of the goggle's expected use altitude. Accordingly, when the goggle is transported to or worn at the goggle's expected use altitude, the pressure within the semi-annular space may be approach, equal or drop below the atmospheric pressure of the goggle's expected use altitude to minimize a pressure difference between the pressure within the semi-annular space and the atmospheric pressure for reducing or eliminating distortion of objects viewed through the goggle. Further, although the pressures discussed herein are in relation to altitudes relating to Mammoth Mountain, such pressures are provided by way of example and not limitation. As such, the pressures may be varied and variously employed to accommodate other ski resorts and other elevations.

Referring now to FIG. 7, atmospheric pressure at sea level may be about 1013.25 mbars. As altitude increases, atmospheric pressure decreases. For example, the atmospheric pressure at an altitude of about two thousand (2000) meters may be about 750 mbars to about 800 mbars which is less than the approximate atmospheric pressure at sea level.

The dual lens assembly 6 may be assembled at sea level. At assembly, the pressure within the semi-annular space may be about equal to the atmospheric pressure at sea level. Dry dehumidified air may be introduced into the semi-annular space, as discussed above. Furthermore, the pressure within the semi-annular space may be set to a level greater than the atmospheric pressure of the goggle's expected use altitude but less than the atmospheric pressure of the goggle's assembly altitude. For example, during assembly of the dual lens assembly 6 at sea level, the pressure within the semi-annular space may be about 1013.25 mbars. The absolute pressure within the space between the lenses 10, 12 may be reduced to a level below the atmospheric pressure at sea level (i.e., goggle's assembly altitude). Accordingly, there is a pressure differential between the atmospheric pressure and the pressure within the semi-annular space that distorts the lenses 10, 12 and objects viewed therethrough. When the goggle 2 is transported to an altitude above sea level (e.g., skiing altitudes, etc.), the pressure within the semi-annular space approaches the atmospheric pressure of the goggle's then current altitude to minimize or eliminate the pressure differential, the distortion of the lenses 10, 12 and the distortion of the objects viewed through the lenses 10, 12.

For an expected use altitude of between about twenty four hundred meters to about thirty three hundred meters, the pressure within the semi-annular space may be set to between about 700 mbars and about 900 mbars (i.e., approximate atmospheric pressure at an altitude of about two thousand meters), and more preferably, to between about 750 mbars to about 800 mbars if the goggle 2 is assembled at about sea level. In this example, the pressure within the semi-annular space was set to a pressure closer to the atmospheric pressure of the goggle's expected use altitude rather than sea level.

At the assembly altitude, there may be a pressure difference between the atmospheric pressure and the pressure within the semi-annular space distorting the lenses 10, 12. Fortunately, when the goggle is transported to its use altitude, the pressure within the semi-annular space approaches or equals the atmospheric pressure of the goggle's expected use altitude when the goggle is brought up to such altitude minimizing the distortion of the lenses 10, 12.

As the pressure within the semi-annular space approaches the atmospheric pressure, the distortion of the lenses 10, 12 is reduced or eliminated. In particular, at assembly, there may be a significant pressure differential between the pressure within the semi-annular space and the atmospheric pressure. The pressure difference may be about 260 mbars to about 310 mbars. The pressure difference may cause the outer and inner lenses to flex such that objects viewed through the dual lens assembly 6 may appear distorted at the assembly altitude. Fortunately, the goggles 2 are typically not worn at the assembly altitude. Rather, the goggles 2 are worn at a higher expected use altitude. As the user drives from a mountain base to a ski resort, the altitude of the goggle 2 increases, the atmospheric pressure decreases, the lenses 10, 12 flex in response to the pressure changes, and the pressure within the semi-annular space may be reduced slightly. Once the user reaches the ski resort, the pressure within the semi-annular space may be about equal to the atmospheric pressure at the ski resort. Now there is less or negligible pressure difference between the atmospheric pressure and the pressure within the semi-annular space. As a result, the lenses 10, 12 will not be significantly distorted and objects viewed through the dual lens assembly 6 will also not be significantly distorted.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A dual pane lens assembly adapted to be installed into a frame of a goggle, the dual pane lens assembly comprising:
an outer lens;
an inner lens positioned proximate the outer lens; and
a gasket interposed between the outer and inner lenses forming a water tight and an air tight semi-annular space therebetween, a pressure within the semi-annular space being below an atmospheric pressure of an assembly altitude of the goggle for reducing distortion due to a pressure difference between the pressure within the semi-annular space and an atmospheric pressure of an assembly altitude of the goggle when the goggle is transported to the goggle's expected use altitude from the goggle's assembly altitude.

2. The dual pane lens assembly of claim 1, wherein the semi-annular space is pressurized to a pressure greater than an atmospheric pressure of the goggle's expected use altitude but less than the atmospheric pressure of the goggle's assembly altitude.

3. The dual pane lens assembly of claim 1 wherein the pressure within the semi annular space is closer to an atmospheric pressure of the goggle's expected use altitude than the atmospheric pressure of the goggle's assembly altitude.

4. The dual pane lens assembly of claim 1, wherein the semi-annular space is pressurized to a pressure equal to about an atmospheric pressure of the goggle's expected use altitude.

5. The dual pane lens assembly of claim 1, wherein the semi-annular space is pressurized to a pressure lower than an atmospheric pressure of the goggle's expected use altitude.

6. The dual pane lens assembly of claim 1 wherein the goggle's expected use altitude is between about twenty four hundred meters to about thirty three hundred meters.

7. The dual pane lens assembly of claim 1 wherein the semi-annular space is pressurized to be between about 700 mbars to about 900 mbars.

8. The dual pane lens assembly of claim 7 wherein the semi annular space is pressurized to be between about 750 mbars to about 800 mbars.

9. The dual pane lens assembly of claim 1 wherein the gasket is a polyurethane based rubber.

10. The dual pane lens assembly of claim 1 wherein the gasket is a polyurethane based glue.

11. The dual pane lens assembly of claim 1 wherein the gasket is self sealing.

12. The dual pane lens assembly of claim 1 wherein the gasket is adhered to the inner lens and the outer lens.

13. A method of fabricating a goggle resistant to fogging; the method comprising the steps of:
a) interposing a gasket between an inner lens and an outer lens of the goggle;
b) forming a water tight and an air tight seal between the inner and outer lenses of the goggle with the gasket;
c) inserting a needle of a syringe through the gasket and into a space between the inner and outer lenses of the goggle;
d) evacuating the air from the space between the inner and outer lenses of the goggle with the syringe;
e) removing the needle from the gasket.

14. The method of claim 13 wherein the gasket is self sealing and the gasket self seals after the needle is removed from the gasket.

15. The method of claim 13 wherein the evacuating step removes substantially all of the moisture from the space between the inner and outer lenses.

16. The method of claim 15 further comprising the step of introducing dry dehumidified air into the space between the inner and outer lenses with a syringe.

17. The method of claim 13 further comprising the step of pressurizing the space between the inner and outer lenses below an atmospheric pressure of an assembly altitude of the goggle.

18. The method of claim 17 further comprising the step of pressurizing the space between the inner and outer lenses to about an atmospheric pressure of an expected use altitude of the goggle.

19. A method of wearing a dual lens goggle, the method comprising the steps of:
a) providing a dual lens goggle with an airtight seal formed between lenses of the goggle;
b) storing the dual lens goggle at a storage altitude which is below a use altitude of the goggle wherein the lenses of the goggle are distorted due to a pressure difference between an atmospheric pressure of the goggle's storage altitude and an absolute pressure within the space between the lenses;
c) transporting the goggle to the goggle's use altitude such that the absolute pressure within the space between the lenses approaches an atmospheric pressure of the goggle's use altitude; and
d) maintaining the airtight seal; and
e) wearing the dual lens goggle.
